(19) United States
(12) Patent Application Publication
(10) Pub. No.: US 2014/0127291 A1
(43) Pub. Date: May 8, 2014

(54) EXTENDED RELEASE ACETAMINOPHEN LIQUID PHARMACEUTICAL COMPOSITIONS

(71) Applicant: Angelo Mario Morella, Athelstone (AU)

(72) Inventor: Angelo Mario Morella, Athelstone (AU)

(73) Assignee: MAYNE PHARMA INTERNATIONAL PTY LTD, Melbourne, Victoria (AU)

(21) Appl. No.: 13/982,179

(22) PCT Filed: Jan. 25, 2012
(86) PCT No.: PCT/AU12/00060

§ 371 Date: Dec. 9, 2013

(30) Foreign Application Priority Data
Jan. 27, 2011 (AU) 2011900305

(51) Int. Cl. A61K 9/50; A61K 31/167

(52) U.S. Cl. CPC A61K 9/5047; A61K 31/167
USPC 424/458; 424/457; 514/629

(57) ABSTRACT

Disclosed herein is an extended release liquid pharmaceutical composition comprising extended release particles dispersed in a liquid medium. The extended release particles comprise acetaminophen dispersed or encapsulated in a water insoluble polymer and/or wax to provide an extended release dosage form of acetaminophen. The liquid medium comprises water, an active pharmaceutical ingredient in immediate release form and, optionally, excipients. The composition is storage stable for at least 6 months at 40° C. and 75% relative humidity.

This patent application claims priority from Australian Provisional Patent Application No. 2011900305 titled “Extended Release Acetaminophen Liquid Pharmaceutical Compositions” and filed 27 Jan. 2011, the entire contents of which are hereby incorporated by reference.

### FIELD

The present invention relates to extended release liquid pharmaceutical compositions of acetaminophen. The present invention also relates to processes for preparing the composition and methods of treatment using the compositions.

### BACKGROUND

The maintenance of desired therapeutic levels of a drug in a patient over time is important. Drugs with relatively short half lives require frequent dosing which increases the likelihood of patient non-compliance, which in turn reduces the effectiveness of therapy or, at the very least, is an inconvenience for patients. For example, acetaminophen (also commonly referred to as “paracetamol” or “APAP”) is a widely used analgesic and antipyretic that has a half life of 1 to 4 hours. As a result, a patient may have to take up to six doses per day in order to maintain effective therapeutic levels.

Extended release formulations of drugs offer some reprieve from the frequent dosing required with short half life drugs. These formulations can maintain therapeutically effective drug levels for longer periods than with immediate release formulations and patient compliance and convenience and, therefore, therapeutic outcomes are improved.

Extended release formulations for oral administration are typically solid dosage forms in which the active ingredient is embedded in a wax or polymer matrix or is encapsulated within a wax or polymer shell. When the solid dosage form comes in contact with surrounding media in vivo the drug diffuses out through the matrix or the shell. The extended release formulation provides levels of drug in the blood that are above the minimum effective therapeutic concentration for an extended period of time which decreases the frequency of administration, thereby improving patient compliance and therapeutic effectiveness.

Oral extended release solid dosage forms, such as tablets or capsules, are difficult to administer to infants and children as their size may make them difficult to swallow. Moreover, many adults also find it difficult to ingest solid oral dose forms. For this reason, there is a need for alternative oral formulations that are more amenable to administration to these patient groups. Liquid formulations of active pharmaceutical ingredients such as solutions or suspensions are widely used for the administration of drugs to children. Suspensions, such as acetaminophen suspensions, typically comprise particles of the active pharmaceutical ingredient suspended in a carbohydrate loaded aqueous solution and they provide immediate release of the active ingredient upon administration. As such, these formulations suffer from the problems of other immediate release formulations in that frequent dosing is required which may lead to reduced compliance and reduced efficacy. Moreover, the carbohydrates used in these formulations, such as sorbitol, mannitol and maltitol, can have undesirable side effects such as a laxative effect when administered up to six times a day. Therefore, reducing the dosing frequency of these liquid formulations may also have the benefit of reducing the amount of the carbohydrates administered to a patient, thereby minimising the undesirable side effects.

Extended release liquid dosage forms formed by adding known extended release solid dosage forms, such as particles, to a liquid medium would not be expected to be stable because the integrity of the polymer coating or wax in or on the particles would be expected to be compromised upon continued exposure to the liquid. Specifically, it would be expected that the active ingredient would diffuse out of the particles into the liquid medium over time. An example of an attempt at such a formulation is disclosed in international patent applications WO 2005/044230 and WO 2005/044246. These patent applications disclose liquid formulations containing immediate release ibuprofen as well as controlled release particles containing ibuprofen. The controlled release ibuprofen particles are coated with a combination of an enteric polymer and a water insoluble polymer and the pH of the liquid medium is less than the pKa of ibuprofen in order to prevent the ibuprofen from diffusing out of the controlled release particles. Notably, there is no data to show that the formulation disclosed in the patent applications is stable for any period of time and nor is there any suggestion or teaching on how to formulate extended release forms of non-acidic drugs whereby control of the pH of the liquid medium cannot be used to prevent or minimise diffusion of the active ingredient out of the extended release particles.

The storage stability of any liquid extended release formulation is paramount. Diffusion of the active ingredient into the liquid medium decreases the storage stability. No data showing storage stability is provided in WO 2005/044230 and WO 2005/044246.

There is a need for extended release liquid dosage forms of acetaminophen that overcome one or more of the problems associated with known formulations.

### SUMMARY

The present invention arises from the surprising finding that extended release particles of acetaminophen are storage stable in a liquid medium.

In a first aspect the present invention provides an extended release liquid pharmaceutical composition comprising extended release particles dispersed in a liquid medium, the extended release particles comprising acetaminophen dispersed or encapsulated in a water insoluble polymer and/or wax to provide an extended release dosage form of acetaminophen, and the liquid medium comprising water, an active pharmaceutical ingredient in immediate release form and, optionally, excipients.

The liquid pharmaceutical composition may be in the form of a suspension, elixir or liquid. Alternatively, the liquid pharmaceutical composition may be formed by reconstituting a dried powder composition with water or an aqueous solution.

In some embodiments, the water insoluble polymer is ethylcellulose.

In some embodiments, the active pharmaceutical ingredient is acetaminophen. These embodiments provide an immediate release/extended release liquid dosage form of acetaminophen that overcomes one or more of the problems associated with prior art dosage forms.

In a second aspect, the present invention provides a method of treating pain or fever in a patient in need of analgesic or antipyretic treatment, the method comprising administering a therapeutically effective amount of the composition of the first aspect of the invention.

In a third aspect, the present invention provides a use of the composition of the first aspect of the invention in the treatment of pain or fever.

In a fourth aspect, the present invention provides a use of the composition of the first aspect of the invention in the preparation of a medicament for the treatment of pain or fever.

### BRIEF DESCRIPTION OF THE ACCOMPANYING FIGURES

FIG. 1 shows the pre-storage dissolution profile of a composition of the present invention comprising a 50:50 mixture of immediate release/extended release acetaminophen immediately after preparation of the composition.

FIG. 2 shows the pre-storage dissolution profile of a composition of the present invention comprising a 75:25 mixture of immediate release/extended release acetaminophen immediately after preparation of the composition.

FIG. 3 shows dissolution profiles of a composition of the present invention comprising a 50:50 mixture of immediate release/extended release acetaminophen immediately after preparation (i.e. pre-storage) (♦); after storage for 1 month in room temperature conditions (25° C. and 60% relative humidity) (▪); and after storage for 2 months in room temperature conditions (25° C. and 60% relative humidity) (▴).

FIG. 4 shows dissolution profiles of a composition of the present invention comprising a 75:25 mixture of immediate release/extended release acetaminophen immediately after preparation (i.e. pre-storage) (♦); after storage for 1 month in room temperature conditions (25° C. and 60% relative humidity) (▪); and after storage for 2 months in room temperature conditions (25° C. and 60% relative humidity) (▴).

FIG. 5 shows dissolution profiles of a composition of the present invention comprising a 50:50 mixture of immediate release/extended release acetaminophen immediately after preparation (i.e. pre-storage) (♦) and after storage at 40° C. and 75% relative humidity for 1 month (▪); after storage at 40° C. and 75% relative humidity for 3 months (▴); and after storage at 40° C. and 75% relative humidity for 6 months (*).

### DETAILED DESCRIPTION

The present invention provides an extended release liquid pharmaceutical composition. As used herein, the term “liquid composition” means a composition that is able to flow at room temperature and includes solutions, suspensions and emulsions. The composition comprises extended release particles dispersed in a liquid medium. The particles may be suspended in the liquid medium. The term “extended release” means any release profile other than a rapid, unrestricted release (i.e. immediate release). Extended release profiles include sustained release, controlled release, multiphase release, retarded release, and the like. Typically, the extended release can take place for several hours to a day.

The extended release particles comprise acetaminophen dispersed or encapsulated in a water insoluble polymer and/or wax to provide an extended release dosage form of acetaminophen. The extended release particles may be in any suitable form provided the acetaminophen is dispersed in the water insoluble polymer and/or wax and/or encapsulated in, the water insoluble polymer and/or wax such that its release from the particles is extended in vivo.

In some embodiments, the extended release particles comprise a core element comprising acetaminophen and a substantially continuous coating of a water insoluble polymer on the core element. The amount of water insoluble polymer used to coat the core element is sufficient to prevent release of the acetaminophen from the core element into the liquid medium when the extended release particles are stored in the liquid medium. In some embodiments, the extended release particles comprise about 20% to about 95%, by weight (based on the total weight of the particle) of the core element and about 5% to about 80%,-by weight of the coating. In some embodiments, the extended release particles comprise about 90% to about 77%, by weight (based on the total weight of the particle) of the core element and about 10% to about 23%, by weight of the coating. In some embodiments, the extended release particles comprise less than about 23% by weight of the coating.

It will be appreciated that the amount of coating required depends on the final particle size, the larger the particle size the less the amount of coating required. The extended release particles have a particle size in the range of about 50 microns to about 2000 microns. In these embodiments, the extended release particles comprise about 50%, by weight (based on the total weight of the particle) of the core element and about 50%, by weight of the coating or about 98%, by weight (based on the total weight of the particle) of the core element and about 2%, by weight of the coating, respectively.

The core element may be of any suitable size. In some embodiments, the size of the core element is from about 10 microns to about 2000 microns. In some embodiments, the core element has a particle size distribution with a median of about 100 microns. In some specific embodiments, the particle size of the extended release particles (i.e. core element and coating) is 35 to 125 microns. In some specific embodiments, the extended release particles are in the form of a fine, non-gritty powder having a particle size distribution with a median of about 100 microns. In some other specific embodiments, the extended release particles are in the form of particles having a particle size distribution with a median of about 300 microns.

The core element may include up to 100% by weight of acetaminophen. Optionally, the core element may also include carriers or excipients, fillers, flavouring agents, stabilising agents and/or colourants. Suitable fillers may be selected from insoluble materials such as silicon dioxide, titanium dioxide, talc, alumina, starch, kaolin, polacrilin potassium, powdered cellulose, microcrystalline cellulose, waxes, and mixtures thereof. Soluble fillers may be selected from mannitol, sucrose, lactose, dextrose, sodium chloride, sorbitol, and mixtures thereof. The filler may be present in amounts of up to approximately 75% by weight based on the total weight of the core element.

In some embodiments, the water insoluble polymer is selected from the group consisting of ethyl cellulose; acrylic and/or methacrylic ester polymers; cellulose acetates; cellulose butyrates; cellulose propionates; poly methacrylates; copolymers of acrylates or methacrylates; and copolymers of acrylates or methacrylates having a low quaternary ammonium content; copolymers of ethyl acrylate, methyl methacrylate containing quaternary ammonium groups; or aqueous dispersions of any of the aforesaid polymers. In specific embodiments, the water insoluble polymer is ethyl cellulose. Notably, the water insoluble polymer coating does not need to contain an enteric, water soluble or reversed enteric polymer or mixtures thereof, although it is contemplated that in some embodiments these polymers could be included in relatively small amounts that do not affect the stability of the extended release particles in the liquid medium.

The water insoluble polymer coating may also comprise a plasticiser. In some embodiments, the plasticiser is selected from the group consisting of: diethyl phthalate, triethyl citrate, triethyl acetyl citrate, triacetin, tributyl citrate, polyethylene glycol, propylene glycol, glycerol, dibutylsebacate, and castor oil. In some embodiments, the plasticiser is present in amounts from 0% to about 50% by weight based on the total weight of the water insoluble polymer coating.

In some embodiments, the thickness of the water insoluble polymer coating is from about 0.005 microns to about 100 microns. In some specific embodiments, the thickness of the coating is from about 0.05 microns to about 5 microns.

The solvent which may be used in the preparation of the coating may be an organic solvent. The solvent may be one in which the water insoluble polymer is soluble but in which acetaminophen is poorly soluble or insoluble. In some embodiments, the solvent for the coating is selected from the group consisting of alcohols such as methanol, ethanol; halogenated hydrocarbons such as dichloromethane; hydrocarbons such as methylcyclohexane; and mixtures thereof. In some specific embodiments, the solvent is dichloromethane.

Suitable extended release particles are disclosed in published United States patent application 20030064108 the details of which are incorporated herein by reference.

In some embodiments, the water insoluble coating is applied to the core elements by spray drying. However, the person skilled in the art will appreciate that a number of other known methods could also be used to apply the coating onto the core element could also be used. For example, acetaminophen crystals could be coated using a fluid bed. Alternatively, the core elements could be coated by coacervation whereby acetaminophen is dispersed in a solution of the coating polymer and a solvent and then phase separated by changing the temperature of the polymer solution, adding a salt, adding a non-solvent, adding incompatible polymer to the polymer solution or inducing a polymer-polymer interaction.

As an alternative to a water insoluble polymer coating, the extended release particles may comprise acetaminophen dispersed or encapsulated in a wax. Any of the pharmaceutically acceptable waxes that are known in the art could be used. Examples include carnauba wax, glyceryl monostearate, shellac wax, paraffin-type waxes, yellow wax, white wax, microcrystalline wax, emulsifying wax, and mixtures thereof. In these embodiments, the extended release particles may be formed by melting the wax material at temperature higher than the melting point of the wax, adding acetaminophen powder to the melted wax and mixing followed by cooling and sieving.

In the compositions of the present invention the extended release particles are suspended or otherwise dispersed in a liquid medium comprising water, an active pharmaceutical ingredient in immediate release form and, optionally, excipients.

As mentioned, we have surprisingly found that the liquid pharmaceutical composition in which the extended release particles are suspended in the liquid medium is stable for at least six months under room temperature conditions. By “stable” we mean that the dissolution profile of the composition after storage is within about 40% of the initial dissolution profile obtained as soon as practicable after preparation of the composition. In some embodiments, the dissolution profile of the composition after storage is within about 30% of the initial dissolution profile obtained as soon as practicable after preparation of the composition. In some embodiments, the dissolution profile of the composition after storage is within about 20% of the initial dissolution profile obtained as soon as practicable after preparation of the composition. In some embodiments, the dissolution profile of the composition after storage is within about 10% of the initial dissolution profile obtained as soon as practicable after preparation of the composition.

By “room temperature conditions” we mean 25° C. and 60% relative humidity. Stability testing under accelerated storage conditions has also shown that the composition is stable for at least six months when stored at 40° C. and 75% relative humidity.

These stability results are surprising as the person skilled in the art would not expect that extended release particles could be maintained in a water medium without substantial dissolution of the acetaminophen from the particles. Dissolution of the acetaminophen from the extended release particles results in a reduction in the amount of acetaminophen available in an extended release form which means that the extended release characteristics of the composition will change as a result. Leakage of the acetaminophen from the extended release particles would have been expected because it would have been thought that the integrity of the water insoluble polymer and/or wax coating would be reduced over time as a result of the constant exposure to water. In addition, it is possible that Ostwald ripening of the extended release particles could result in depletion of acetaminophen from the extended release particles or conversely cause the core element to grow and consequently rupture the coating.

The liquid medium comprises water, an active pharmaceutical ingredient and, optionally excipients.

Various excipients suitable for use in liquid dosage forms are known in the art and can be used in the liquid medium. For example, sweeteners, surfactants, buffers, preservatives, solubilisers, emulsifiers, bacteriostatic agents, colorants, and flavours can be used. The amounts of each excipient can readily be determined or ascertained by the person skilled in the art.

Examples of suitable sweeteners include sucrose, dextrose, high fructose corn syrup, maltose, sorbitol, xylitol, mannitol, maltitol, sucralose, aspartame, saccharin, acesulfam K, and the like. In some embodiments, the composition includes a sweetener selected from one or more of the group consisting of: sorbitol, mannitol, maltitol and saccharin. In some embodiments, the sweetener is a mixture of sorbitol, mannitol and saccharin.

Examples of suitable preservatives include esters of p-hydroxybenzoic acid, such as sodium methyl hydroxybenzoate and sodium propyl hydroxybenzoate.

The liquid medium may also contain other water-miscible liquid diluents. Many such liquid diluents are known in the art and include, for example, glycols, alcohols and glycerol. However, the amount of liquid diluent used must be low enough so that the water insoluble polymer and/or wax does not dissolve.

In some embodiments, the liquid medium comprises water, the active pharmaceutical ingredient in immediate release form, one or more sweeteners, and one or more preservatives. In some of these embodiments, the one or more sweeteners comprise a mixture of sorbitol, maltitol and saccharin sodium. In some of these embodiments, the one or more preservatives comprise a mixture of sodium methyl hydroxybenzoate and sodium propyl hydroxybenzoate.

In some embodiments, the liquid medium comprises: from about 0.001% to about 20% w/w of the active pharmaceutical ingredient in immediate release form; from about 0% to about 80% w/w sweetener; from about 0% to about 1% w/w of preservatives; optionally, other excipients, with the balance being water.

The liquid medium may be any suitable pH within a range that is physiologically and organoleptically acceptable. There is no need to adjust or regulate the pH of the liquid medium to obtain product stability in the case of the compositions of the present invention. In other words, the liquid composition of the present invention does not require the use of pH regulating agents. Acetaminophen can be considered to be neutral at physiologically and organoleptically acceptable pH. However, pH regulating agents could be used in some cases, such as to match a particular flavour (e.g. acidic for citrus flavour). If pH regulating agents are added to the liquid medium, the pH is preferably from about 4 to about 7.

The composition may comprise the active pharmaceutical ingredient in immediate release form and acetaminophen in extended release forms in a ratio of about 10:90 to about 90:10. In some specific embodiments, the composition comprises a 50:50 mixture of the active pharmaceutical ingredient in immediate release form and acetaminophen in extended release form. In some other specific embodiments, the composition comprises a 75:25 mixture of the active pharmaceutical ingredient in immediate release form and acetaminophen in extended release form.

In some embodiments, the active pharmaceutical ingredient in immediate release form is acetaminophen. This provides an immediate release/extended release liquid dosage form of acetaminophen that is suitable for use by infants, children and older persons unable to swallow tablets and which requires less frequent dosing than currently available liquid dosage forms of acetaminophen.

In some embodiments, the composition comprises acetaminophen in immediate release form and extended release forms in a ratio of about 10:90 to about 90:10. In some embodiments, the composition comprises acetaminophen in immediate release form and extended release forms in a ratio of about 20:80 to about 80:20. hi some specific embodiments, the composition comprises a 50:50 mixture of acetaminophen in immediate release form and extended release form. In some other specific embodiments, the composition comprises a 75:25 mixture of acetaminophen in immediate release form and extended release form.

In some embodiments, the concentration of active pharmaceutical ingredient in immediate release form in the liquid medium exceeds the saturation concentration for the active ingredient. In the case of acetaminophen, the saturation concentration in water is about 14 mg/ml and, therefore, in these embodiments, the concentration of acetaminophen in immediate release form in the liquid medium is greater than about 1.4% by weight.

Thus, in some embodiments, the present invention provides an extended release liquid pharmaceutical composition comprising extended release particles dispersed in a liquid medium, the extended release particles comprising acetaminophen dispersed or encapsulated in ethylcellulose to provide an extended release dosage form of acetaminophen, and the liquid medium comprising water, acetaminophen in immediate release form, sweeteners, preservatives and, optionally, favours, thickeners and colours.

In some other embodiments, the active pharmaceutical ingredient in immediate release form is a non-steroidal anti-inflammatory drug (NSAID). In some embodiments, the NSAID is a propionic acid derivative. Examples of propionic acid derivatives include ibuprofen, naproxen, benoxaprofen, naproxen sodium, fenbufen, flurbiprofen, fenoprofen, fenbuprofen, ketoprofen, indoprofen, pirprofen, carpofen, oxaprofen, pranoprofen, microprofen, tioxaprofen, suprofen, alminoprofen, tiaprofenic acid, fluprofen, bucloxic acid, and pharmaceutically acceptable salts, derivatives, and combinations thereof. In some embodiments, the propionic acid derivative is ibuprofen.

In some other embodiments, the active pharmaceutical ingredient in immediate release form is an opioid analgesic. Examples of opioid analgesics include codeine, dihydrocodeine, hydrocodone, oxycodone, and pharmaceutically acceptable salts, derivatives, and combinations thereof.

In some other embodiments, the active pharmaceutical ingredient in immediate release form is an antitussive. Examples of antitussives include dextromethophan, codeine, pholcodine, and pharmaceutically acceptable salts, derivatives, and combinations thereof.

In some other embodiments, the active pharmaceutical ingredient in immediate release form is an antihistamine. Examples of antihistamines include bilastine, cetirizine, desloratidine, fexofenadine, loratadine, levocitirizine, chlorpheniramine, brompheniramine, diphenhydramine, diphenylpyraline, mequitazine, alimemazine, and pharmaceutically acceptable salts, derivatives, and combinations thereof.

In some other embodiments, the active pharmaceutical ingredient in immediate release form is a calmative. Examples of calmatives include doxylamine and pharmaceutically acceptable salts and derivatives thereof.

In some other embodiments, the active pharmaceutical ingredient in immediate release form is a muscle relaxant. Examples of muscle relaxants include orphenadrine and pharmaceutically acceptable salts and derivatives thereof.

In some other embodiments, the active pharmaceutical ingredient in immediate release form is a mucolytic. Examples of mucolytics include acetylcysteine, guaifenesin, ambroxol, bromhexine, carbocisteine, and pharmaceutically acceptable salts, derivatives, and combinations thereof.

In some other embodiments, the active pharmaceutical ingredient in immediate release form is a sympathomimetic. Examples of sympathomimetics include pseudoephedrine, phenylephrine, and pharmaceutically acceptable salts, derivatives, and combinations thereof

The extended release liquid pharmaceutical composition of the present invention will typically be in the form of a suspension. However, it is also contemplated that the liquid pharmaceutical composition could be formed by reconstituting a dried powder using water or an aqueous solution. In this case, the composition may be stable for at least one week once reconstituted.

The present invention also provides a method of treating pain or fever in a patient in need of treatment, the method comprising administering a therapeutically effective amount of the composition of the first aspect of the invention.

The term “therapeutically effective amount” or “effective amount” means an amount sufficient to effect beneficial or desired clinical results. An effective amount can be administered in one or more administrations. An effective amount is typically sufficient to palliate, ameliorate, stabilise, reverse, slow or delay the progression of the disease state.

The composition may be provided to a patient in need of treatment in a single administration that provides for the release of the acetaminophen in the blood over about a 6 hour period, an 8 hour period, a 12 hour period, or more.

In a third aspect, the present invention provides a use of the composition of the first aspect of the invention in the treatment of pain or fever.

In a fourth aspect, the present invention provides a use of the composition of the first aspect of the invention in the preparation of a medicament for the treatment of pain or fever.

The invention is hereinafter described by way of the following non-limiting examples.

### EXAMPLES

### Example 1

### Materials

Extended Release Acetaminophen Particles

Microencapsulated acetaminophen was produced as follows. In the following amounts, ethyl cellulose was dissolved in dichloromethane and acetaminophen powder was then dispersed in the solution to produce a slurry.

| | |
|---|---|
| Ethyl cellulose | 7% w/w |
| Acetaminophen | 28% w/w |
| Dichloromethane | 65% w/w |

The slurry was then spray dried to produce extended release acetaminophen particles in the form of a white, free flowing powder consisting of 80% w/w acetaminophen and 20% w/w ethyl cellulose with a median particle size of less than 150 μm.

Liquid Medium Containing Acetaminophen

An aqueous suspension of acetaminophen available as “Children's Panadol Colourfree Suspension (5-12 years)” was used as purchased. Each mL of the suspension contained:
Acetaminophen (48 mg)
Sodium methyl hydroxybenzoate
Sodium propyl hydroxybenzoate
Saccharin sodium
Sorbitol (155 mg)
Mannitol (265 mg)
Water

### Example 2

### Dissolution Testing

Dissolution experiments were performed using compositions containing 250 mg-280 mg acetaminophen in 900 mL of pH 5.8 media at 37° C. using USP Apparatus II (Paddle Method) at 100 rpm. The dissolution experiments were carried out in accordance with accepted methods as provided in USP 23/NF 18, United States Pharmacopeial Convention, Inc., Rockville, Md. 1791 (1994).

Samples were removed at various time points, filtered, and the quantity of dissolved acetaminophen in the filtrate determined by measuring the UV absorbance of samples at 290 nm.

### Example 3

### Preparation of Composition and Dissolution Testing

An aqueous suspension of acetaminophen and extended release particles of acetaminophen (prepared as described in Example 1) were combined to give a 50:50 immediate release/extended release acetaminophen suspension (“Mixture 1”) and a 75:25 immediate release/extended release acetaminophen suspension (“Mixture 2”) as outlined in Table 1.

TABLE 1

| | Mixture 1 (50:50) | Mixture 2 (75:25) |
|---|---|---|
| Acetaminophen suspension | 100 mL | 100 mL |
| (48 mg/mL) | | |
| Extended release | 6.0 g | 2.0 g |
| acetaminophen particles | | |

The mixtures were visually examined for homogeneity and were deemed to be homogeneous.

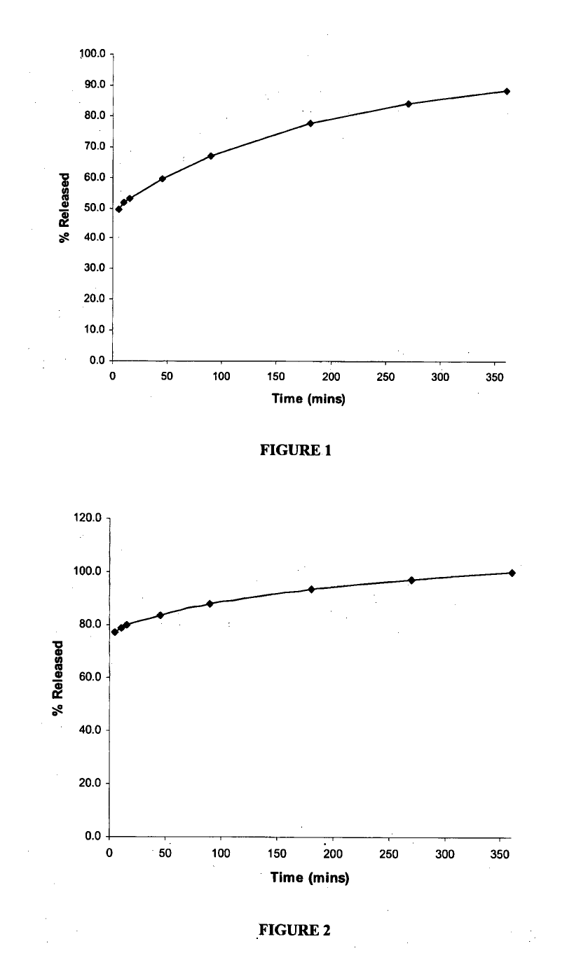

Dissolution testing of the mixtures was then carried out according to Example 2. Both mixtures 1 and 2 were adequately dispersed throughout the dissolution media. The dissolution profiles of mixtures 1 and 2 are shown in FIGS. 1 and 2 respectively, which demonstrate the rapid dissolution of acetaminophen over 5 to 10 minutes due to the immediate release component and sustained release of acetaminophen due to the extended release microcapsules over 6 hours.

### Example 4

### Room Temperature Stability Testing

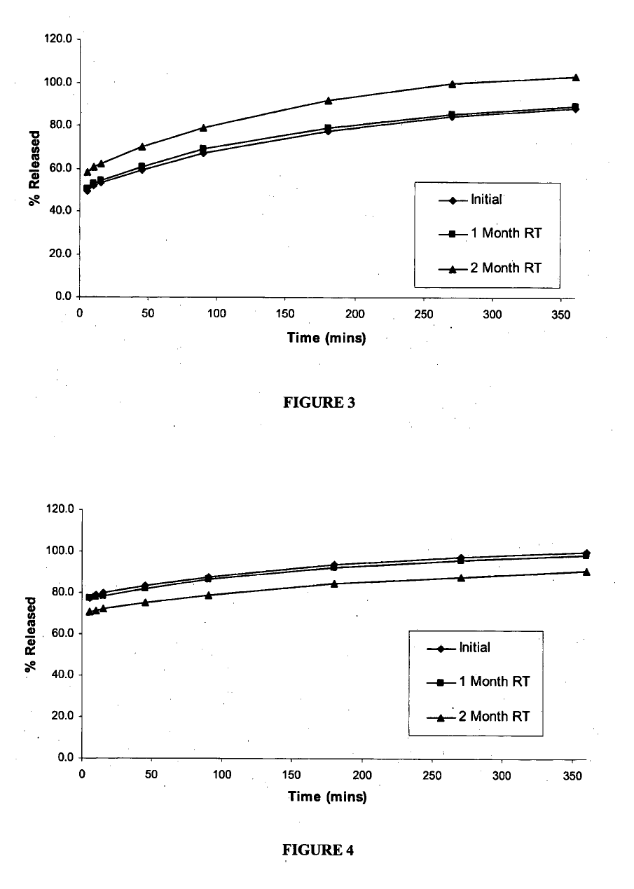

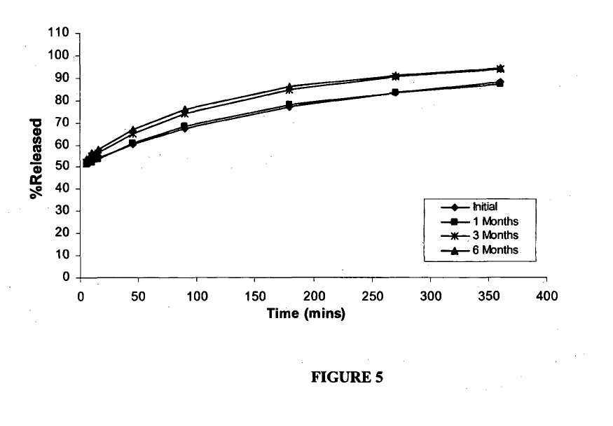

The stability of Mixture 1 and Mixture 2 after storage over 2 months was determined under room temperature conditions (i.e. 25° C./60% relative humidity) as defined in CPMP/ICH/2736/99 (ICH Q1A (R2) “Stability Testing Guidelines: Stability Testing of New Drug Substances and Products”, The European Agency for the Evaluation of Medicinal Products, London, 2003). The resultant dissolution profiles are shown in FIGS. 3 and 4.

Over the 2 month trial, no obvious separation of layers or solidification occurred. The mixtures could be stirred easily, so any settling of the extended release acetaminophen particles to give an inhomogeneous mixture could be rectified by stirring or shaking.

After 1 month in room temperature conditions, the post-storage dissolution profiles of the mixtures compared well with the initial (pre-storage) profiles. After 2 months in room temperature conditions, the post-storage dissolution profiles differed by ˜10-20% from the initial (pre-storage) profiles.

### Example 5

### Accelerated Stability Testing

The stability of Mixture 1 was determined under “accelerated” storage conditions as defined in PCMP/ICH/2736/99 (ICH Q1A (R2) “Stability Testing Guidelines: Stability Testing of New Drug Substances and Products”, The European Agency for the Evaluation of Medicinal Products, London, 2003). Mixture 1 was stored in a sealed bottle at 40° C./75% relative humidity for 6 months. The resultant dissolution profiles after 1, 3 and 6 months storage are shown in FIG. 5.

The data show that the amount of acetaminophen released at any time on the 6 month storage dissolution profile is within about 20 percentage points of the amount of acetaminophen released at any time on the initial (pre-storage) dissolution profile. Indeed, the amount of acetaminophen released at many time points on the 6 month storage dissolution profile is within about 10 percentage points of the amount of acetaminophen released at the equivalent time point on the initial (pre-storage) dissolution profile.

In conclusion, we have shown that the release of acetaminophen from compositions according to the present invention is extended beyond that achieved with an immediate release acetaminophen suspension alone. Therefore, compositions according to the present invention may provide good antipyretic and analgesic control over longer periods than immediate release acetaminophen alone. A particularly beneficial result of this is maintenance of analgesic and antipyretic effects overnight whilst reducing the disruption of sleep due to the need for repeated administration of immediate release acetaminophen suspension.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. An extended release liquid pharmaceutical composition comprising extended release particles dispersed in a liquid medium, the extended release particles comprising acetaminophen dispersed or encapsulated in a water insoluble polymer and/or wax to provide an extended release dosage form of acetaminophen, and the liquid medium comprising water, an active pharmaceutical ingredient in immediate release form and, optionally, excipients.

2. The extended release composition of claim 1, wherein upon in vitro dissolution testing, the amount of acetaminophen and/or active pharmaceutical ingredient released at any time point on a post-storage dissolution profile is within about 40 percentage points of the amount of acetaminophen and/or active pharmaceutical ingredient released at the equivalent time point on a pre-storage dissolution profile.

3. The extended release composition of claim 1, wherein upon in vitro dissolution testing, the amount of acetaminophen and/or active pharmaceutical ingredient released at any time point on a post-storage dissolution profile is within about 30 percentage points of the amount of acetaminophen and/or active pharmaceutical ingredient released at the equivalent time point on a pre-storage dissolution profile.

4. The extended release composition of claim 1, wherein upon in vitro dissolution testing, the amount of acetaminophen and/or active pharmaceutical ingredient released at any time point on a post-storage dissolution profile is within about 20 percentage points of the amount of acetaminophen and/or active pharmaceutical ingredient released at the equivalent time point on a pre-storage dissolution profile.

5. The extended release composition of claim 1, wherein upon in vitro dissolution testing, the amount of acetaminophen and/or active pharmaceutical ingredient released at any time point on a post-storage dissolution profile is within about 10 percentage points of the amount of acetaminophen and/or active pharmaceutical ingredient released at the equivalent time point on a pre-storage dissolution profile.

6. The extended release composition of claim 1, wherein the water insoluble polymer is ethylcellulose or cellulose acetate.

7. The extended release composition of claim 1, wherein the extended release particles comprise acetaminophen dispersed or encapsulated in a wax.

8. The extended release composition of claim 1, wherein the extended release particles comprise a core element comprising acetaminophen and a substantially continuous coating of the water insoluble polymer and/or wax on the core element.

9. The extended release composition of claim 8, wherein the extended release particles comprise about 20% to about 95%, by weight (based on the total weight of the particles) of the core element and about 5% to about 80%, by weight of the coating.

10. The extended release composition of claim 9, wherein the extended release particles comprise less than about 23% by weight of the coating.

11. The extended release composition of claim 1, wherein the liquid medium contains a sweetener selected from one or more of the group consisting of: sorbitol, mannitol, and maltitol.

12. The extended release composition of claim 1, wherein the liquid medium comprises: from about 0.001% to about 20% of the active pharmaceutical ingredient in immediate release form; from about 0% to about 80% sweetener; from about 0% to about 1% of preservatives; optional excipients; with the remainder being water.

13. The extended release composition of claim 12, wherein the active pharmaceutical ingredient in immediate release form is acetaminophen.

14. The extended release composition of claim 13, wherein the composition comprises acetaminophen in immediate release form and extended release form in a ratio of from about 10:90 to about 90:10.

15. The extended release composition of claim 14, wherein the composition comprises a 50:50 mixture of acetaminophen in immediate release form and extended release form.

16. The extended release composition of claim 14, wherein the composition comprises a 75:25 mixture of acetaminophen in immediate release form and extended release form.

17. The extended release composition of claim 1, wherein the composition is provided to a patient in need of treatment in a single administration that provides for the release of the acetaminophen in the blood over a period of at least 6 hours.

18. A method of treating pain or fever in a patient in need of treatment, the method comprising administering a therapeutically effective amount of the composition of claim 1.

19. Use of the composition of claim 1 in the treatment of pain or fever.

20. (canceled)
